(19) United States
(12) Patent Application Publication
(10) Pub. No.: US 2026/0317705 A1
(43) Pub. Date: Oct. 15, 2026

(54) VASCULARIZED CENTRAL NERVOUS SYSTEM (CNS) ORGANOIDS AND METHODS OF PREPARING THE SAME

(71) Applicant: Drexel University, Philadelphia, PA (US)

(72) Inventors: Liang Oscar Qiang, Philadelphia, PA (US); Victor Ogbolu, Philadelphia, PA (US)

(21) Appl. No.: 19/644,819

(22) Filed: Apr. 10, 2026

Related U.S. Application Data
(60) Provisional application No. 63/787,075, filed on Apr. 11, 2025.

(51) Int. Cl. C12N 5/0793; C12N 5/071; C12N 5/079

(52) U.S. Cl. CPC C12N 5/0619; C12N 5/0608; C12N 5/0622; C12N 5/069; C12N 2501/11; C12N 2501/113; C12N 2501/115; C12N 2501/155; C12N 2501/165; C12N 2501/999; C12N 2502/04; C12N 2506/45; C12N 2513/00

(57) ABSTRACT

Described herein is a method of preparing an organoid. The method comprises preparing a central nervous system (CNS) organoid by culturing first stem cells under a neuronal differentiation condition for a first duration of time; preparing a mesoderm organoid by culturing second stem cells under a mesodermal differentiation condition for a second duration of time; dissociating the cells in the CNS organoid to obtain a plurality of dissociated CNS cells; and co-culturing the plurality of dissociated CNS cells with the mesoderm organoid under a co-culture condition. Also described herein is an organoid, which comprises neurons or neural progenitor cells (NPCs); microglia cells; and a microvascular network. The organoid is immunocompetent.

### CROSS-REFERENCE TO RELATED APPLICATIONS

The present application claims priority under 35 U.S.C. § 119(e) to U.S. Provisional Patent Application No. 63/787,075, filed Apr. 11, 2025, which is incorporated herein by reference in its entirety.

### BACKGROUND

Recent advances in central nervous system (CNS) organoids have transformed region-specific modeling of human neurodevelopment and neurological disease. However, conventional organoids lack integrated microvasculature and microglia, substantially limiting their physiological fidelity and their capacity to interrogate neurovascular and neuroimmune crosstalk.

There is a need for CNS organoids that mimic real CNS tissues in a more physiologically accurate and relevant manner, such as CNS organoids that contain microvasculature and/or microglia. The present invention addresses this need.

### SUMMARY

In some aspects, the present invention is directed to the following non-limiting embodiments:

### Method of preparing organoid and organoid prepared using the same

In some aspects, the present invention is directed to a method of preparing an organoid.

In some embodiments, the method comprises preparing a central nervous system (CNS) organoid by culturing first stem cells under a neuronal differentiation condition for a first duration of time. In some embodiments, the CNS organoid comprises neural progenitor cells, neurons, or astrocytes.

In some embodiments, the method further comprises preparing a mesoderm organoid by culturing second stem cells under a mesodermal differentiation condition for a second duration of time.

In some embodiments, the method further comprises dissociating the cells in the CNS organoid to obtain a plurality of dissociated CNS cells.

In some embodiments, the method further comprises co-culturing the plurality of dissociated CNS cells with the mesoderm organoid under a co-culture condition.

In some embodiments, the first stem cells and the second stem cells are the same type of stem cells (syngeneic).

In some embodiments, the first stem cells and the second stem cells are from the same stem cell source.

In some embodiments, the first stem cells or the second stem cells are independently induced pluripotent stem cells (iPSCs), optionally wherein the first stem cells or the second stem cells are independently human iPSCs (hiPSCs).

In some embodiments, the first duration of time is longer than the second duration of time.

In some embodiments, the first duration of time ranges from about 18 days to about 24 days.

In some embodiments, the second duration of time ranges from about 10 days to about 15 days.

In some embodiments, co-culturing the plurality of dissociated CNS cells with the mesoderm organoid comprising co-culturing a predetermined number of the dissociated CNS cells and a predetermined number of the mesoderm organoid, such that the ratio between the dissociated CNS cells and the mesoderm organoid allows ratios between neural progenitors, astrocytes, blood vessel endothelial cells, and microglia cells are similar to the ratios in a brain tissue from an in vivo source.

In some embodiments, co-culturing the plurality of dissociated CNS cells with the mesoderm organoid comprising co-culturing about 80,000 to about 150,000 dissociated CNS cells with one mesoderm organoid.

In some embodiments, the plurality of dissociated CNS cells and the mesoderm organoid are co-cultured in a biomaterial scaffold.

In some embodiments, the neuronal differentiation condition comprises treatment with dorsomorphin (DM), SB431542, EGF, or FGF.

In some embodiments, the mesodermal differentiation condition comprises treatment with CHIR99021, bFGF, BMP4, or VEGF.

In some embodiments, the co-culture condition comprises treatment with BDNF, NT3, VEGF, IL-34, or M-CSF.

In some aspects, the present invention is directed to a organoid prepared according to the method herein.

### Organoid

In some aspects, the present invention is directed to an organoid.

In some embodiments, the organoid comprises neurons or neural progenitor cells (NPCs).

In some embodiments, the organoid further comprises microglia cells. In some embodiments, the microglia cells comprise TMEM119-expressing cells; and

In some embodiments, the organoid further comprises a microvascular network. In some embodiments, the microvascular network comprises CD31-expressing endothelial cells.

In some embodiments, the microglia cells are immunocompetent.

In some embodiments, the microglia cells are in a resting, ramified state when the organoid is in a homeostatic state.

In some embodiments, the microglia are activated when the organoid is in an inflamed state.

In some embodiments, the microglia are capable of activation when the organoid challenged by oligomeric Tau.

In some embodiments, the microvascular network comprises both endothelial cells and mural cells.

In some embodiments, the organoid comprises a blood brain barrier (BBB).

In some embodiments, the organoid comprises pericytes or cells expressing PDGFRβ along the microvascular network.

In some embodiments, the organoid comprises astrocytes or cells expressing GFAP along the microvascular network.

In some embodiments, the organoid is perfusable.

In some embodiments, all or some of the cells in the organoid are differentiated from induced pluripotent stem cells (iPSCs). In some embodiments, all the cells in the organoid are differentiated from a single source of iPSCs.

In some embodiments, the iPSCs are human iPSCs (hiPSCs).

In some embodiments, the amount of the SOX2⁺ cells ranges from about 5% to about 70% based on a total number of cells.

In some embodiments, the area occupied by the GFAP⁺ cells ranges from about 10% to about 80% based on a total area of the organoid.

In some embodiments, the area occupied by the TMEM119⁺ cells ranges from about 2% to about 10% based on a total area of the organoid,

In some embodiments, the CD31⁺ vessel‑like area ranges from about 5% to about 20% based on a total area of the organoid.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1D illustrate certain aspects of the optimization of biomaterial scaffolds in viCOs, in accordance with some embodiments. (FIG. 1A) viCOs cultured with a Matrigel scaffold (FIG. 1B) exhibit increased CD31 expression compared to viCOs without Matrigel (FIG. 1A), accompanied by a higher number of CD31⁺ vessel branches (FIG. 1C) and increased total vessel length (FIG. 1D)

FIGS. 2A-2D illustrate certain aspects of the optimization of cortical organoid cell number in viCOs, in accordance with some embodiments. One‑month‑old viCOs show the percentage of (FIG. 2A) SOX2⁺ neural progenitors, (FIG. 2B) GFAP⁺ astrocytes, (FIG. 2C) CD31⁺ vessel‑like structures, and (FIG. 2D) TMEM119⁺ cells

FIGS. 3A-3P illustrate certain aspects of the characterization of syngeneic vascularized, immune-competent cortical organoids, in accordance with some embodiments. FIGS. 3A-3B: one-month organoids show neurons, ECs, and neural progenitor cells. FIG. 3C: By 2 months, mature astrocytes emerge and associate with ECs. FIGS. 3D-3J: Smooth muscle cells and pericytes are incorporated into vessel-like structures. FIGS. 3K-3L: Microglia detected by TMEM119 and IBA1. FIGS. 3M-3P: qRT-PCR validates EC and microglia integration in vascularized immune-competent cortical organoids (viCOs), using non-vascularized cortical organoids (hCO).

FIGS. 4A-4G provide representative tissue-clearing and lightsheet images, in accordance with some embodiments. FIGS. 4A-4B: IMARIS 3D reconstruction shows lumenized, branched vasculature. FIG. 4C: Cross-section highlights vascular lumen. FIGS. 4D-4G: Quantification of lumen diameter, branching points, vessel length, and segment number using IMARIS analysis.

FIG. 5 demonstrates that single-cell RNA sequencing reveals diverse cell populations in vascularized, immune-competent cortical organoids (viCOs), in accordance with some embodiments. Uniform manifold approximation and projection (UMAP) visualization of viCOs, colored by cell type annotations derived from established marker gene expression.

FIGS. 6A-6D demonstrate that the organoids herein are a useful model for studying abhorrent protein aggregation, commonly found in e.g. neurodegenerative diseases, in accordance with some embodiments. Oligomeric tau (oTau) seeding in vascularized, immune-competent cortical organoids induces extensive intracellular tau pathology and microglial activation. oTau seeding induces robust tau pathology detected by AT8, TNT2, and TOC1 antibodies. FIG. 6H: Microglial activation shown by IBA1+ microglia, with shift from ramified to amoeboid morphology.

FIGS. 7A-7B provide schematics of generating region-specific vascularized, immune-competent cortical organoid, in accordance with some embodiments.

FIGS. 8A-8C demonstrate that the vascularized, immune-competent cortical organoid expresses CCL2, CXCL8, and CXCL10 when challenged by oTau, in accordance with some embodiments.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

The present study developed a method of preparing vascularized CNS organoids comprising microvascular networks, thus mimicking in vivo CNS tissues.

Specifically, the present study discovered that co-culturing dissociated CNS organoid cells with dissociated mesoderm organoid cells, and co-culturing intact CNS organoids with dissociated mesoderm organoid cells all failed to result in desirable CNS organoids with microvascular networks. Surprisingly, co-culturing dissociated CNS organoid cells with intact mesoderm organoids successfully achieved vascularized CNS organoids.

The present study further discovered that varying the ratios between the dissociated CNS organoid cells and intact mesoderm organoids can alter the composition of the cell type in the obtained vascularized CNS organoids, allowing compositions similar to in vivo CNS tissues to be achieved. In addition, the use of biomaterial scaffolds in the co-culture step of the dissociated CNS organoid cells and the intact mesoderm organoids can further improve the microvascular network in the vascularized CNS organoids.

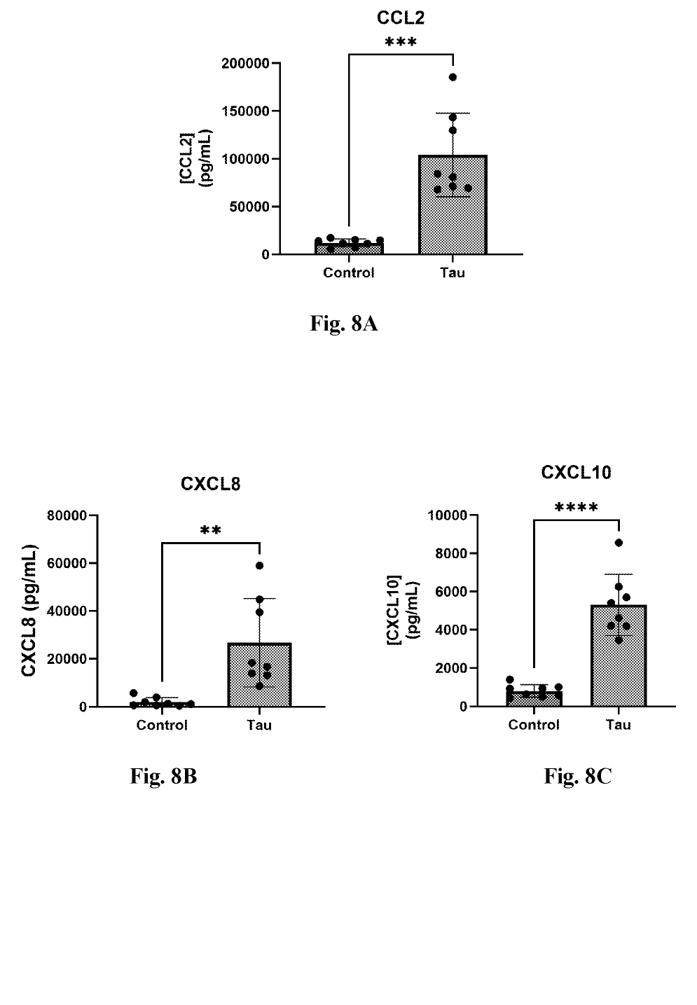

It is worth noting that the vascularized CNS organoids contemplated herein contain microglia cells, and that these microglia cells are immunocompetent and can produce inflammatory response in response to, e.g., the challenge of toxic intermediate of protein aggregation such as oligomeric Tau. Alphalisa data further demonstrate that vascularized CNS organoids express CCL2, CXCL8, and CXCL10, indicating a reactive, pro-inflammatory state. (See e.g., FIGS. 8A-8C. Brain-specific microglia express CXCL8, CXCL10, and CCL2 when they transition into a highly active, proliferative state that directly alters the local brain microenvironment.)

Accordingly, in some aspects, the present invention is directed to a method of preparing an organoid, such as a CNS organoid, such as a vascularized CNS organoid or an immunocompetent CNS organoid.

In some aspects, the present invention is directed to an organoid, such as a CNS organoid, such as a vascularized CNS organoid or an immunocompetent CNS organoid.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

### Methods of Preparing Organoids and Organoids Prepared Using the Same

In some aspects, the present invention is directed to a method of preparing an organoid.

In some embodiments, the organoid is a vascularized central nervous system (CNS) organoid. In some embodiments, the organoid is a vascularized and immunocompetent CNS organoid. In some embodiments, the organoid is an immunocompetent CNS organoid.

In some embodiments, the organoid has various features that make it desirable for mimicking CNS tissues obtained from an in vivo source such as a brain of a subject. Non-limiting examples of such features are described elsewhere herein.

### Preparing CNS organoid

In some embodiments, the method comprises preparing a central nervous system (CNS) organoid by culturing first stem cells under a neuronal differentiation condition for a first duration of time. In some embodiments, the CNS organoid comprises neural progenitor cells, neurons, and astrocytes.

The methods of preparing CNS organoids are known in the art, and are described in, for example, Mohan, N., et al. (Modeling spastic paraplegia 4 with corticospinal motor neuron-enriched cortical organoids reveals genotype-phenotype and HDAC6-targetable pathology. Cell Reports, 2026. 45(3)), Sun, X., et al. (Vascularized Brain Assembloids With Enhanced Cellular Complexity Provide Insights Into the Cellular Deficits of Tauopathy. Stem Cells, 2024. 42(2): p. 107-115), and Yates, P.L., et al. (Veteran-derived cerebral organoids display multifaceted pathological defects in studies on Gulf War Illness. Frontiers in Cellular Neuroscience, 2022. Volume 16 – 2022). .

In some embodiments, the first duration of time is about 17 days, about 18 days, about 19 days, about 20 days, about 21 days, about 22 days, about 23 days, about 24 days, about 25 days, or any ranges therebetween.

In some embodiments, the neuronal differentiation condition used to prepare the CNS organoid the stem cells comprises treatment with dorsomorphin (DM), SB431542, EGF, and/or FGF.

### Preparing mesoderm organoid

In some embodiments, the method further comprises preparing a mesoderm organoid by culturing second stem cells under a mesodermal differentiation condition for a second duration of time.

The methods of preparing mesoderm organoids are known in the art, and are described in, for example, Miao, Y., et al. (Co-development of mesoderm and endoderm enables organotypic vascularization in lung and gut organoids. Cell, 2025. 188(16): p. 4295-4313.e27) and Wimmer, R.A., et al. (Human blood vessel organoids as a model of diabetic vasculopathy. Nature, 2019. 565(7740): p. 505-510).

In some embodiments, the second duration of time is about 9 days, about 10 days, about 11 day, about 12 days, about 13 days, about 14 days, about 15 days, about 16 days, or any ranges therebetween.

In some embodiments, the first duration of time (for culturing the first stem cells into the CNS organoid) is longer than the second duration of time (for culturing the second stem cells into the mesoderm organoid).

In some embodiments, the mesodermal differentiation condition used to prepare the mesoderm organoid from the second stem cells comprises CHIR99021, bFGF, BMP4, and/or VEGF.

### Dissociating CNS organoid

In some embodiments, the method further comprises dissociating the cells in the CNS organoid to obtain a plurality of dissociated CNS cells.

In some embodiments, the cells in the CNS organoid are dissociated enzymatically. Non-limiting examples of enzymes suitable for dissociating cells in the CNS organoid include trypsin, collagenase, dispase, papain, elastase, accutase, DNase, and the like.

### Co-culturing dissociated CNS cells with mesoderm organoid

In some embodiments, the method further comprises co-culturing the plurality of dissociated CNS cells with the mesoderm organoid under a co-culture condition.

The present study discovered, in one aspect, that use of certain ratios between the dissociated CNS cells and the mesoderm organoids during the co-culturing step result in ratios between neural progenitors, astrocytes, blood vessel endothelial cells, and/or microglia cells similar to those in actual in vivo CNS tissues. This allows for preparing vascularized CNS organoids more similar to real CNS tissues from subjects. Accordingly, in some embodiments, the dissociated CNS cells and the mesoderm organoids are mixed in predetermined ratios to achieve such desirable results.

In some embodiments, about 50,000, about 60,000, about 70,000, about 80,000, about 90,000, about 100,000, about 110,000, about 120,000, about 130,000, about 140,000, about 150,000, or about 160,000 dissociated CNS cells (or any ranges therebetween) are mixed with every one mesoderm organoid in the co-culturing step.

The present study discovered, in one aspect, that co-culturing the dissociated CNS cells and the mesoderm organoid in a biomaterial scaffold improves the formation of the microvascular network in the organoid. Accordingly, in some embodiments, the plurality of dissociated CNS cells and the mesoderm organoid are co-cultured in a biomaterial scaffold.

Non-limiting embodiments of biomaterial scaffolds include natural polymer hydrogels such as Matrigel (a gelatinous protein mixture secreted by Engelbreth-Holm-Swarm (EHS) mouse sarcoma cells, which include laminin, collagen IV, heparan sulfate proteoglycans, among others), collagen hydrogels (such as collagen type I hydrogels), hyaluronic acid (HA)-based hydrogels, alginate-based hydrogels, gelatin methacryloyl (GelMA) and the like.

In some embodiments, the co-culture condition used to co-culture the dissociated CNS cells and the mesoderm organoid comprises treatment with BDNF, NT3, VEGF, IL-34, and/or M-CSF.

In some embodiments, the first stem cells and the second stem cells are the same type of stem cells (for example, the first stem cells and the second stem cells are both induced pluripotent stem cells (iPSCs), or are both embryonic stem cells (ESCs)).

In some embodiments, the first stem cells and the second stem cells are from the same stem cell source (for example, the first stem cells and the second stem cells are the same established cell line, or are induced to have pluripotency from the same parent cells).

In some embodiments, the first stem cells or the second stem cells are induced pluripotent stem cells (iPSCs).

In some embodiments, the first stem cells or the second stem cells are human iPSCs (hiPSCs).

In some aspects, the present invention is directed to an organoid prepared according to the method herein.

### Organoid

In some aspects, the present invention is directed to an organoid.

In some embodiments, the organoid comprises: neurons or neural progenitor cells (NPCs); microglia cells; and a microvascular network.

In some embodiments, the microglia cells comprise TMEM119-expressing cells

In some embodiments, the microvascular network comprises CD31-expressing endothelial cells

The vascularized CNS organoids generated in the present study include microglia cells. Notably, the vascularized CNS organoids displayed signs of inflammation when challenged with oligomeric Tau, and the microglia cells changed their morphology from the ramified shape indicating resting state to shapes indicating activation. These results indicate that the vascularized CNS organoids are immunocompetent.

Accordingly, in some embodiments, the microglia cells are immunocompetent. In some embodiments, the microglia cells are in a resting, ramified state when the organoid is in a homeostatic state. In some embodiments, the microglia are activated when the organoid is in an inflamed state, optionally the microglia are capable of activation when the organoid challenged by oligomeric Tau.

In the vascularized CNS organoids generated in the present study, multiple cell types found in in vivo CNS microvascular networks were confirmed to be present. Notably, cell types of the blood brain barrier (BBB) in in vivo CNS tissues were also found to be present along the microvascular network in the organoids herein.

Accordingly, in some embodiments, the microvascular network comprises both endothelial cells and mural cells.

In some embodiments, the organoid comprises a blood brain barrier (BBB).

In some embodiments, the organoid comprises pericytes or cells expressing PDGFRβ along the microvascular network.

In some embodiments, the organoid comprises astrocytes or cells expressing GFAP, such as along the microvascular network.

In some embodiments, the organoid herein is perfusable.

In some embodiments, all or some of the cells in the organoid are differentiated from induced pluripotent stem cells (iPSCs), optionally wherein all the cells in the organoid are differentiated from a single source of iPSCs.

In some embodiments, the iPSCs are human iPSCs (hiPSCs).

In some embodiments, the amount of the SOX2⁺ cells in the organoid is about 4%, about 5%, about 6%, about 8%, about 10%, about 15%, about 20%, about 25%, about 30%, about 40%, about 50%, about 60%, about 70%, about 80% based on a total number of cells, or any ranges therebetween.

In some embodiments, the area occupied by the GFAP⁺ cells is about 8%, about 10%, about 15%, about 20%, about 30%, about 40%, about 50%, about 60%, about 70%, about 80%, or about 90% based on a total area of the organoid, or any ranges therebetween.

In some embodiments, the area occupied by the TMEM119⁺ cells is about 1.5%, about 2%, about 3%, about 4%, about 5%, about 6%, about 7%, about 8%, about 9%, about 10%, or about 12% based on a total area of the organoid, or any ranges therebetween.

In some embodiments, the CD31⁺ vessel‑like area is about 4%, about 5%, about 6%, about 7%, about 8%, about 9%, about 10%, about 12%, about 14%, about 16%, about 18%, about 20%, or about 22%, based on a total area of the organoid, or any ranges therebetween.

### Examples

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Vascularized Immunocompetent Cortical Organoid

Recent advances in human induced pluripotent stem cell (hiPSC)-derived central nervous system (CNS) organoids have transformed region-specific modeling of human neurodevelopment and neurological disease. However, conventional organoids lack integrated microvasculature and microglia, substantially limiting their physiological fidelity and their capacity to interrogate neurovascular and neuroimmune crosstalk.

To overcome these constraints, the present study engineered syngeneic vascularized, immunocompetent cortical organoids (viCOs) by incorporating endothelial, mural, and microglial lineages derived from a single hiPSC source into a unified three-dimensional cortical architecture.

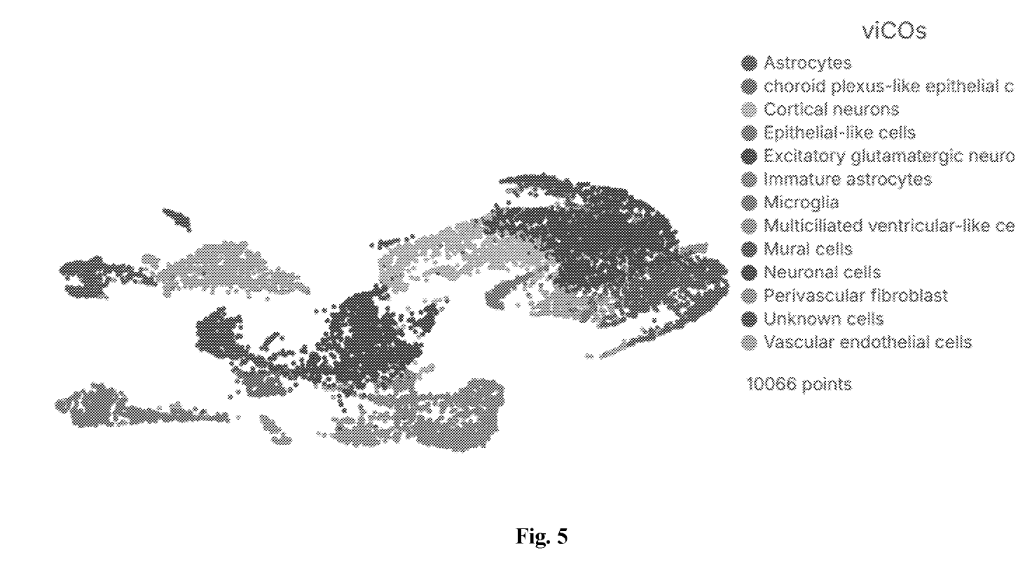

These next-generation constructs establish widely spread endothelial-mural networks that extend throughout the organoid core and incorporate transcriptionally defined, functionally mature microglia, as validated by single-cell RNA sequencing (FIG. 5).

When challenged with AD-patient-derived tau fibrils, viCOs recapitulated core features of human tauopathy, including tau hyperphosphorylation and aggregation, microglial activation, and amplified inflammatory signaling. Notably, the system revealed neurodegeneration.

By integrating neuronal, vascular, and immune compartments within a human 3D microphysiological framework, viCOs transcend key limitations of existing organoid models and establish an improved platform to dissect the reciprocal interactions among tau pathology, neurovascular compromise, and inflammation, with broad implications for mechanistic discovery and therapeutic innovation in AD and AD-related dementias.

### Example 2

The recent advancement of human induced pluripotent stem cell (hiPSC) technology has revolutionized disease modeling, offering a versatile platform to generate various human central nervous system (CNS) cells from patient-derived sources. This provides a human specific platform to probe cell-specific mechanisms involved in disease progression. While these platforms have significantly advanced the ability to model disease, they primarily rely on two-dimensional (2D) cultures with minimal co-culturing. This limitation restricts opportunities to study complex cell-cell interactions that are crucial in the brain’s three-dimensional environment. Development of 3D brain organoids has provided more physiologically relevant models, better recapitulating the complex cellular interactions and spatial organization found in the human brain. These organoids demonstrate remarkable complexity, exhibiting 3D structures that mirror key features of human brain development. Notably, they contain defined proliferative zones that reflect the multi-layered progenitor zone organization found in the developing human brain. Furthermore, they generate diverse neuronal subtypes, including all six cortical layers, showcasing their capacity for self-organization and differentiation, making them ideal candidates to model disease in a human specific platform.

Recently, there has been a rapid increase in the use of CNS organoids to study neurodegenerative diseases, underscoring their value as human-relevant models that capture aspects of brain architecture and cell–cell interactions not seen in 2D cultures or many animal systems. The transition to 3D hiPSC-derived brain and cortical organoids has produced models that approximate in vivo cytoarchitecture and regional identity, yet they still lack several elements essential for faithfully modeling disease onset and progression. In particular, most current CNS organoids do not incorporate vascular cells, which are crucial for regulating neural stem cell niches, metabolic support, and blood–brain barrier function, or microglia, which are central to tissue homeostasis, synaptic remodeling, and neuroimmune responses. The absence of these components limits the ability of existing models to fully capture key pathological drivers of neurodegenerative disease, including neurovascular defects and inflammation.

Neurodegenerative diseases such as Alzheimer’s disease (AD) comprising aberrant tau conformations, chronic inflammatory cascades, and their combined disruption of neurovascular architecture are increasingly recognized as interconnected drivers of neurodegeneration rather than parallel, independent processes. Pathogenic tau species accumulate not only in neurons but also along the cerebrovasculature, where they contribute to blood–brain barrier (BBB) breakdown, impaired perfusion, and neurovascular defect, thereby amplifying neuronal vulnerability and accelerating disease progression. Recently, it was demonstrated that supplementing organoids with human umbilical vein endothelial cells and myeloid progenitors enhances neuronal maturation and unmasks more robust inflammatory cytokine responses in MAPT (tau) mutant organoids, underscoring how vascular and immune components modulate tau-driven phenotypes.

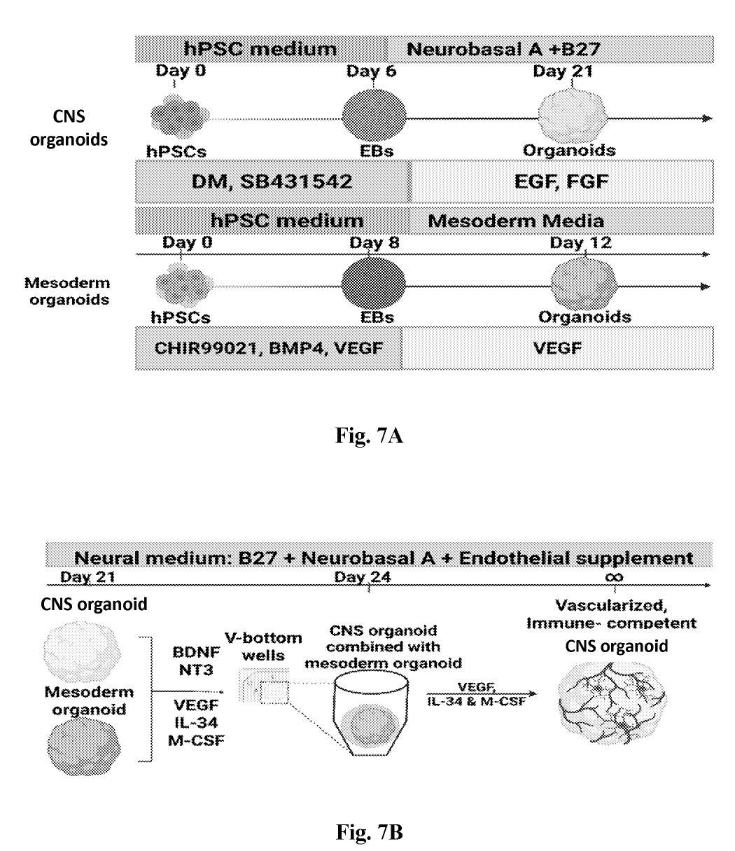

However, these systems often rely on non-isogenic microglia or lack vascular elements and BBB-like properties. Despite these important steps, there is still no integrated, syngeneic human model in which microglia, endothelial cells, mural cells, astrocytes, and neurons all derive from the same hiPSC line, which is crucial for dissecting cell type–specific and genotype dependent contributions to neurodegenerative disease while avoiding confounds from mixed genetic backgrounds. To address these critical gaps, the present study established vascularized human organoid models integrated with microglia, all derived from syngeneic hiPSCs, as a novel approach to model neurodevelopment and neurodegeneration (FIGS. 7A-7B). This strategy leverages advancements in stem cell technology and tissue engineering to create 3D models that closely mimic the structure and function of AD relevant brain regions. By incorporating microglia and a complex vascular network, these organoids promise to provide insights into development and degeneration, exploring the intricate interplay between these various cell types.

### Example 3: Materials and Methods

### Human induced pluripotent stem cell lines

Human induced pluripotent stem cells (hiPSCs) KOLF2.1J cell line derived from a European 55 year old healthy male donor which is commercially available from the Jackson laboratory. (Pantazis, C.B., et al., A reference human induced pluripotent stem cell line for large-scale collaborative studies. Cell Stem Cell, 2022. 29(12): p. 1685-1702.e22).

All hiPSC lines were cultured in 5% CO₂ at 37° C. in mTeSR Plus (Stem Cell Technologies, 100–0276) on Matrigel GFR Basement Membrane Matrix (Corning, 356231). Briefly, 6- well plate (Thermo Scientific, 140675) was coated with Matrigel diluted in ice-cold DMEM-F12 (Gibco, 11330032) for 1 h at 37° C. Once all hiPSCs reach 70% confluence, they are passaged using ReLeSR (Stem Cell Technologies, 100–0483), incubated for 4 min and subsequently plated on coated dishes with 10μM ROCK inhibitor Y-27632 (Tocris, 1254). 3mL Medium changes with mTeSR Plus were performed every other day.

### Cortical organoid differentiation and culture

hiPSCs were cultured on Matrigel-coated 6-well plates with mTeSR Plus medium. The culture was kept in a 37° C. and 5% CO₂ incubator throughout the experiment. On day 0, hPSCs colonies were dissociated to single cells using ACCUTASE™ (Gibco, A1110501) for 5 minutes at 37° C. and then washed with DMEM/F12 medium. Approximately 2.5x10⁶ single hiPSCs were then resuspended in mTeSR Plus medium with 10μM Y-27632, 5μM Dorsomorphin (Sigma, C956T21), and 10μM SB431542 (Tocris, 1614) to promote embryoid bodies (EBs) formation. Single cell hiPSC solution were plated into one well of an AggreWell 800. The AggreWell was rinsed with 500μl of anti-adherence rinsing solution and centrifuged at 1,300 rcf for 5 minutes before cell plating. After plating, cells were incubated in 5% CO₂ at 37° C. for 3 days. on day 3, EBs were transferred to a 6 well ultra-low attachment plate (Corning, 3471). From day 3–6, a half medium changes with Dorsomorphin and SB431542 in mTeSR Plus was performed every other day. From days 7–21, half medium change was performed using neuronal differentiation media I which consist of Neurobasal A (Thermo Fisher, 10-888- 022), B-27 without Vitamin A (Thermo Fisher, 12587010), Glutamax (Thermo Fisher, 35- 050–061), 20ng/mL EGF (PeproTech, AF-100-15) and 20ng/mL bFGF (Peprotech, 100-18B).

### Mesoderm organoid differentiation and culture

To generate mesoderm organoids, hiPSCs were cultured on Matrigel-coated 6-well plates with mTeSR Plus medium. The culture was kept in a 37° C. and 5% CO₂ incubator throughout the experiment. On day 0, hPSCs colonies were dissociated to single cells using ACCUTASE™ (Gibco, A1110501) for 5 minutes at 37° C. and then washed with DMEM/F12 medium. Approximately 2.5x10⁶ single hiPSCs were then resuspended in mTeSR Plus medium with 10μM Y-27632 and 20ng/mL bFGF (Peprotech, 100-18B). On day 3 EBs were transferred into Clinostar bioreactors to ensure consistent size across EBs From day 3-5, the media for embryoid bodies are changed to mTeSR Plus medium supplemented with 3 μm CHIR 99021 (Tocris, 4423). From day 5-8 media was changed to mTeSR Plus medium supplemented with 30ng/ml of BMP4, 20ng/ml (Stemcell technologies, 78211) of bFGF (Peprotech, 100-18B) and VEGF 50 ng/mL (Stemcell technologies, 78073.2). From day 8-12 media was changed to DMEM/F12, supplemented with 50μg/ml of Endothelial cell growth supplement (Corning, 256006), 10% Fetal bovine serum (R&D Systems, S11150), 1X Glutamax (Gibco, 35050061), 1X Non-Essential Amino Acids (Gibco, 1114007), Heparin 50μg/ml (Sigma, H3149), 5uM of SB431542 (Tocris, 1614) and 50 ng/mL of VEGF (Stemcell technologies, 78073.2).

### Generation of vascularized, immunocompetent cortical organoids

Cortical organoids are first dissociated into a single cell suspension via enzymatic dissociation using acutase and DNase. The cells are then resuspended in Neurobasal medium supplemented with B-27, 10μM of the ROCK inhibitor Y-27632, 20ng of BDNF, 20ng NT3, and 10ng of VEGF. These cells are seeded into v-bottom wells at a density of 100k cells per 100μl. Specifically, 50μl of a 50K cell suspension is added into each v-bottom well and incubated for 10 minutes to allow the cells to settle. Next, a mesoderm organoid is added into the same well, followed by another 50μl of the 50K cell suspension. The v-bottom wells are then incubated in a 5% CO₂ incubator at 37° C. for 72 hours for combination. After 72 hours, organoids were removed from the ultra-low attachment plates and embedded in Matrigel.

### Organoid fixation, section and immunohistochemistry (IHC)

Organoids were washed once with PBS (Gibco, 10010023) and fixed in 4% PFA overnight at 4° C. followed by dehydration in 30% sucrose until fully saturated. Organoids were then embedded in M1 Embedding Matrix (Epredia, 1310) and cryosectioned at 25μm thickness and placed on charged slides (Fisher Scientific, 1255016). The slides were dried at room temperature overnight. Organoid mounted slides were then hydrated and washed in 1X PBS 3 times for 5 min followed by quenching (70% PBS, 30% methanol, 0.5% hydrogen peroxide) for 1 h at room temperature. Subsequently, the slides were washed in PBS to remove the quenching buffer and incubated in either donkey (Jackson ImmunoResearch, 017-000-121) or goat (Jackson ImmunoResearch, 005-000-121) serum blocking buffer diluted in PBS for 1 h at room temperature. Then primary antibodies listed in key resource table diluted in PBS and 0.1% Triton X-100 was incubated overnight at 4° C. followed by PBS washes and corresponding secondary antibodies conjugated to Alexa Fluor 488, 555, or 647 incubated for 2 h at room temperature. Lastly, the slides were washed in PBS and incubated with DAPI at 1:20,000 dilution (Fisher Scientific, D1306) and mounted using Fluoro-Gel with Tris Buffer (Electron Microscopy Sciences 1798510). Organoid sections were imaged using Leica SP8 inverted confocal microscope and Leica Thunder microscope. ImageJ was used to measure average fluorescence intensity of each organoid section and individual neuronal processes.

### Single-cell RNA sequencing (scRNA-seq)

10X Chromium Fixed RNA Kit, Human Transcriptome kit and 10X Chromium Controller system was used to generate single cell data. Briefly, 3-month-old organoids were dissociated using 4 mL StemPro Accutase Cell Dissociation Reagent (Thermo Fisher Scientific, A1110501) for 12 min at 37° C. Subsequently, the organoids were quenched and treated briefly with DNAase I (Thermo Fisher Scientific, EN0521) and gently triturated using 2mL serological pipettes to generate single cell suspension in PBS with 1% BSA. Cell viability and count were assessed using trypan blue staining and a hemocytometer. The cell suspension was then loaded onto Chromium next GEM Chip Q (10x Genomics, PN-1000422) per manufacturer instructions, with a targeted cell recovery of 5000–10000 cells. Post GEM formation, the samples were subjected to reverse transcription, amplification, fragmentation and clean-ups, as recommended by the manufacturer. The present study then performed Illumina library preparation using the Dual Index plate TS Set A (10X Genomics, PN-1000251). The subsequent library was then sequenced using NovaSeq6000 at a depth of 25000–30000 paired end reads per cell.

Raw sequencing data were initially processed and aligned to GRCh38 (human) using Cell Ranger Count v7.0.1 (10X Genomics, Cloud analysis) and HDF5 files were generated. The HDF5 files were then uploaded to the Partek Flow software with scRNAseq toolkit (Illumina). Using a single cell QA/QC and a noise-reduction pipeline, the cells were further filtered to exclude low quality cells and features. The filtered set was then normalized using the recommended counts per million, add 1, log 2 transformation workflow. Principal component analysis (PCA) was performed on the filtered cells and further clustered using Louvain graph-based clustering algorithm using the top 20 PCAs and visualized using uniform manifold approximation and projection (UMAP). The present study then extracted the biomarkers for these clusters and used them to manually annotate the clusters. Panglao database, Human protein atlas and extensive literature review were used to annotate clusters. Gene expression profile of various marker genes was generated using the same 2D UMAP as cluster annotations.

### RNA Isolation and qRT-PCR

RNA was isolated from various organoid samples using the PureLink RNA Mini Kit (Thermo Fisher Scientific, 12183018A) and subsequently reverse transcription was performed using the High-Capacity cDNA Reverse Transcription Kit (Thermo Fisher Scientific, 4368813) to synthesize cDNA. Quality control and quantification was performed using the NanoDrop Spectrophotometer. qRT-PCR was performed using the 2X Universal SYBR Green Fast qPCR Mix (ABclonal, RK21203) on the StepOnePlus Real-Time PCR System (Applied Biosystems). RNA primers were acquired from Integrated DNA Technologies.

### Example 4: Generation, optimization and characterization of syngeneic immunocompetent vascularized cortical organoid

The incorporation of microvascular endothelial cells and microglia into the cortical organoids significantly promote their maturity compared to organoids without these cells. Moreover, these immunocompetent vascularized organoids harboring a pathogenic tau mutation demonstrated activated microglial responses and enhanced neuroinflammation phenotypes, characteristics notably absent in conventional organoid models.

Day 21 was chosen as the time point for dissociation and reaggregation of cortical organoids, preserving neuroectodermal integrity and maintaining neural progenitors, neurons, and astrocytes. By day 21, cortical patterning is complete and cultures transition to maturation and maintenance. At this stage, a high proportion of neuronal and glial progenitors renders the organoids sufficiently plastic to survive dissociation and recombination. In contrast, organoids dissociated 7 days earlier or 7 days later fail to successfully recombine, underscoring the critical nature of this window. Having optimized cortical organoid recombination at day 21, the next step was to optimize mesoderm integration parameters, cortical organoid number and the use of ECM matrix-based hydrogels such as Matrigel to promote mesodermal cell development and provide structural support for vessel outgrowth.

Optimization of mesoderm and cortical organoid combination in the viCO model. To determine the optimal integration strategy, three experimental conditions were tested: Condition 1 (dissociated mesoderm + dissociated cortical organoid), Condition 2 (intact mesoderm + dissociated cortical organoid), and Condition 3 (dissociated mesoderm + intact cortical organoid). At one-month post-combination, Condition 2 yielded the best results. Condition 1 failed to properly reaggregate after dissociation and combination, resulting in poor organoid formation. Condition 3, while forming cohesive organoids, lacked mesodermal-derived cell types, suggesting that dissociation of the mesoderm disrupted its developmental potential.

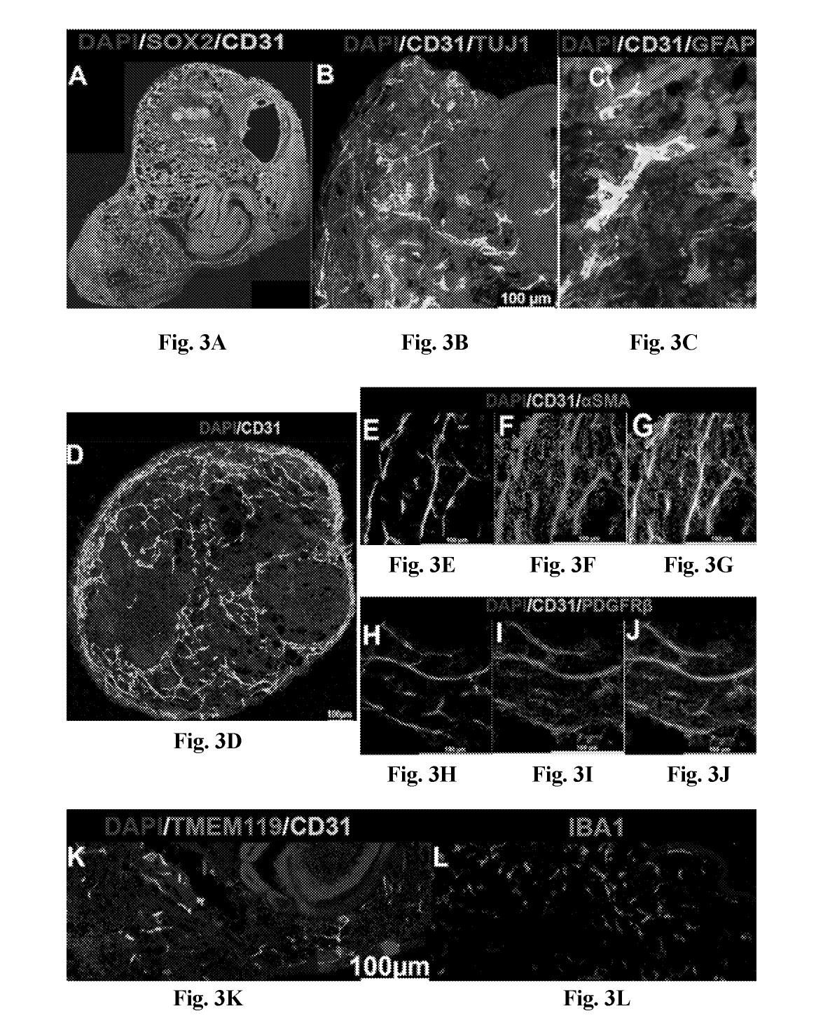

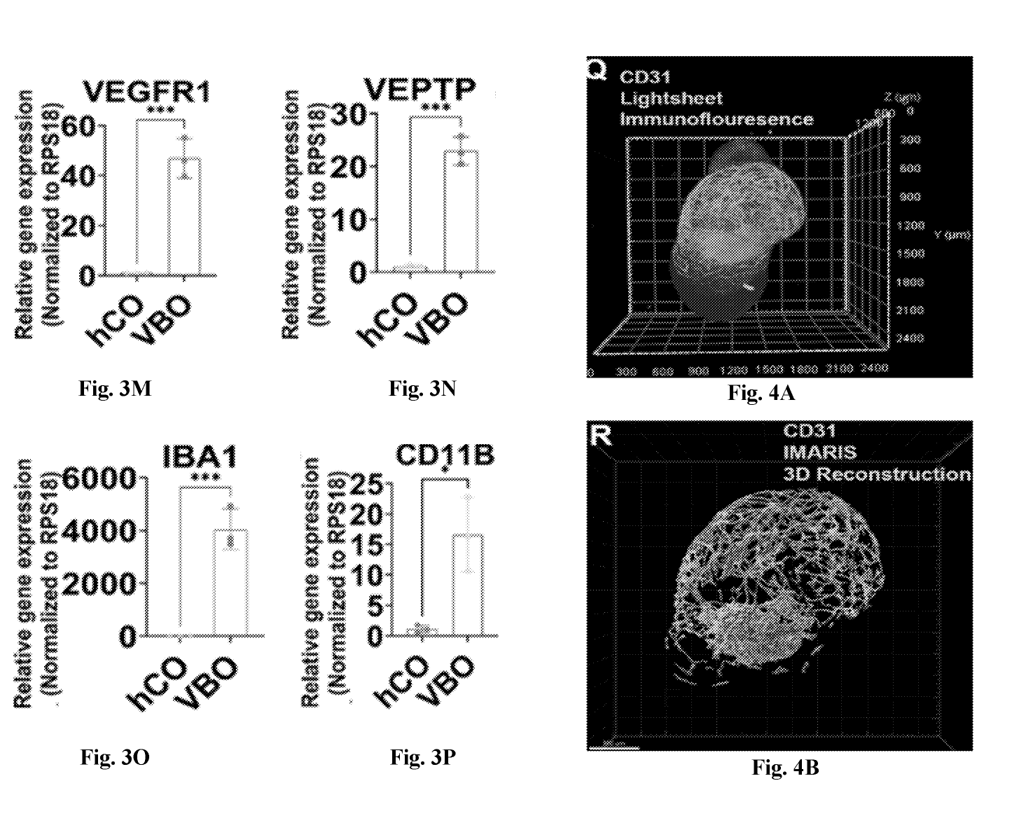

In contrast, Condition 2 demonstrated successful integration of all key mesodermal cell lineages. These organoids exhibited robust CD31+ vessel-like structures, mural cell populations including αSMA+ smooth muscle cells and PDGFRβ+ pericytes, and TMEM119+ microglia (FIGS. 3A-3P). These findings establish Condition 2, combining intact mesoderm organoids with dissociated cortical cells as the preferred approach for generating vascularized, immunocompetent cortical organoids.

### Example 5: Optimization of biomaterial scaffold to enhance angiogenesis in vascularized, immunocompetent organoids

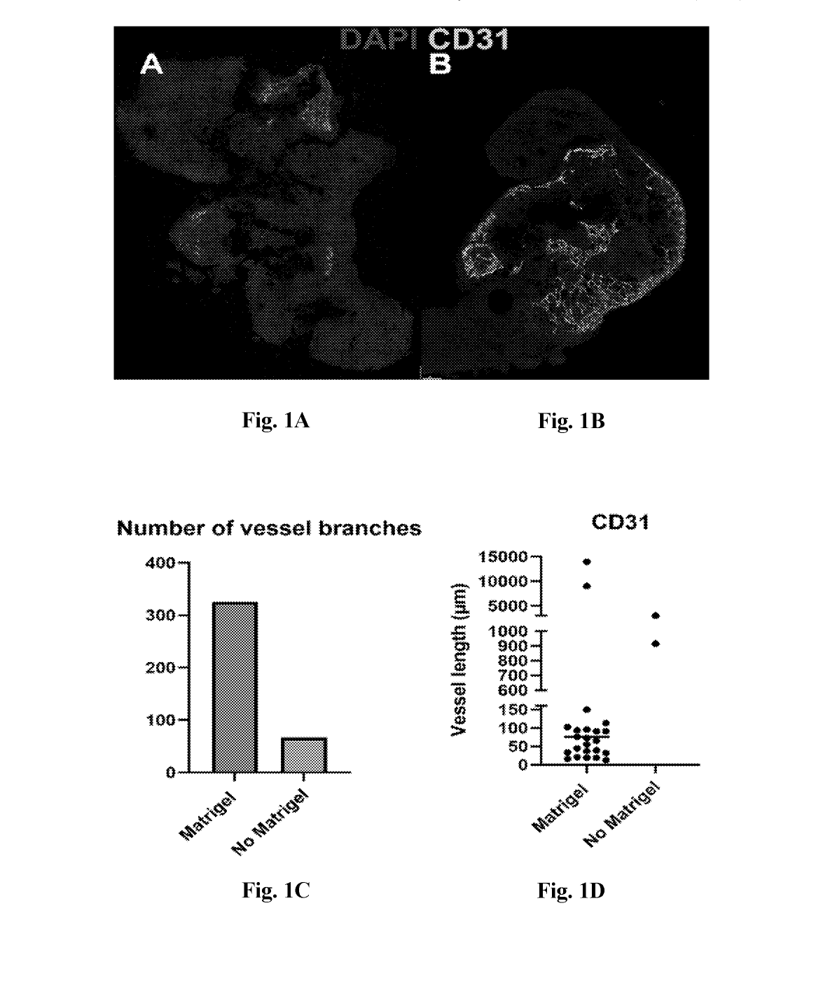

To determine whether extracellular matrix scaffolding enhances vascular development, organoids were embedded in Matrigel following mesoderm-cortical organoid integration. Organoids cultured with Matrigel scaffold exhibited significantly more pronounced angiogenesis, showing a marked increase in CD31+ vessel-like structures compared to viCOs cultured without scaffold support. Quantitative analysis using IMARIS software revealed that Matrigel-embedded organoids displayed increased vessel branching complexity and extended vessel length compared to non-scaffolded controls (FIGS. 1A-1D). Additionally, vessels in scaffolded organoids appeared to penetrate more extensively into the surrounding tissue, suggesting that the biomaterial matrix provides essential biochemical and mechanical cues that promote endothelial cell migration, sprouting, and network formation. These findings demonstrate that Matrigel scaffolding is an effective strategy for enhancing vascularization in viCO models, potentially improving their physiological relevance for studying neurovascular interactions and disease modeling.

### Example 7: Optimization of cortical organoid cell number ratio in vascularized, immune competent organoids

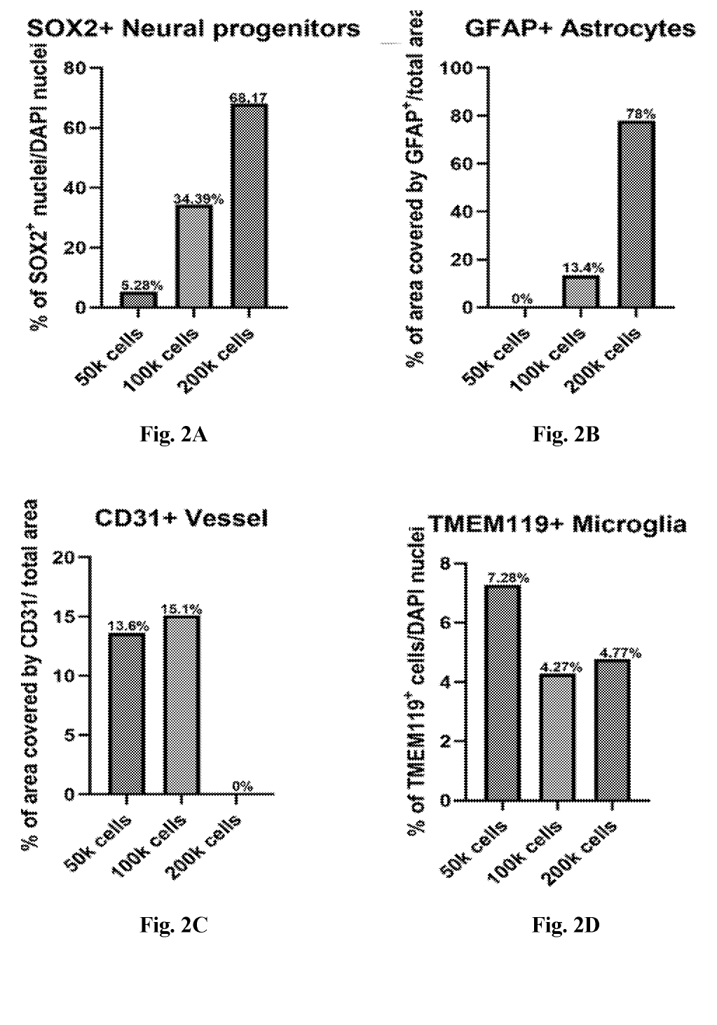

One‑month‑old viCOs were used to determine the optimal initial cortical cell number for balancing neuroectodermal, glial, vascular, and microglial compartments relative to compositions observed in the developing human cortex, where neural progenitors constitute roughly 15–20% of nuclei, glial cells about 15%, microglia ~5%, and microvasculature ~20% of brain volume. (FIGS. 2A-2D) At 50k starting cells, viCOs contained 5.28% SOX2⁺ neural progenitors, 13.4% GFAP⁺ astrocytic area, 13.6% CD31⁺ vessel‑like area, and 7.28% TMEM119⁺ microglia, indicating near representation of neural progenitors, astrocyte and microglia fractions and a marked close representation of vascular structures relative to in vivo levels. At 100k cells, SOX2⁺ progenitors rose to 34.39% of nuclei, GFAP⁺ astrocytes to 13.4% area, CD31⁺ vessels to 15.1% area, and TMEM119⁺ cells decreased to 4.27%, yielding an excess of neuroectodermal and vascular compartments but microglia fraction close to the ~5% developmental benchmark. At 200k cells, SOX2⁺ progenitors reached 68.17% of nuclei and GFAP⁺ astrocytes 78% area, while CD31⁺ vessel‑like structures dropped to 0% and TMEM119⁺ microglia to 4.77%, indicating that high cortical seeding favors progenitor and astrocyte expansion at the expense of vascularization.

### Example 8: Characterization of vascularized, immunocompetent cortical organoid

The organoid model includes a diverse spectrum of CNS cell types, demonstrating the formation of mature cytoarchitecture in prolonged cultures (FIGS. 3A-3O). Indeed, by 3 months in cultures, these organoids exhibited extensive formation of CD31+ endothelial-based microvascular networks with emerging tubular structures (FIGS. 3D-3J), as well as mature microglial marker, TMEM119 (FIG. 3K). The data showed that microglia-like cells constituted approximately 5% of the total cell population in 3-month organoids. IHC analysis revealed abundant vessel-like microvascular structures composed not only of endothelial cells but also accompanied mural cells, such as pericytes and smooth muscle cells labeled by PDGFRβ and αSMA (FIGS. 3E-3J). A significant proportion of these elements maintained close contact with astrocytes (GFAP+), indicating neurovascular unit formation in these organoids.

### Example 9: Vascular architecture in vascularized, immunocompetent cortical organoid

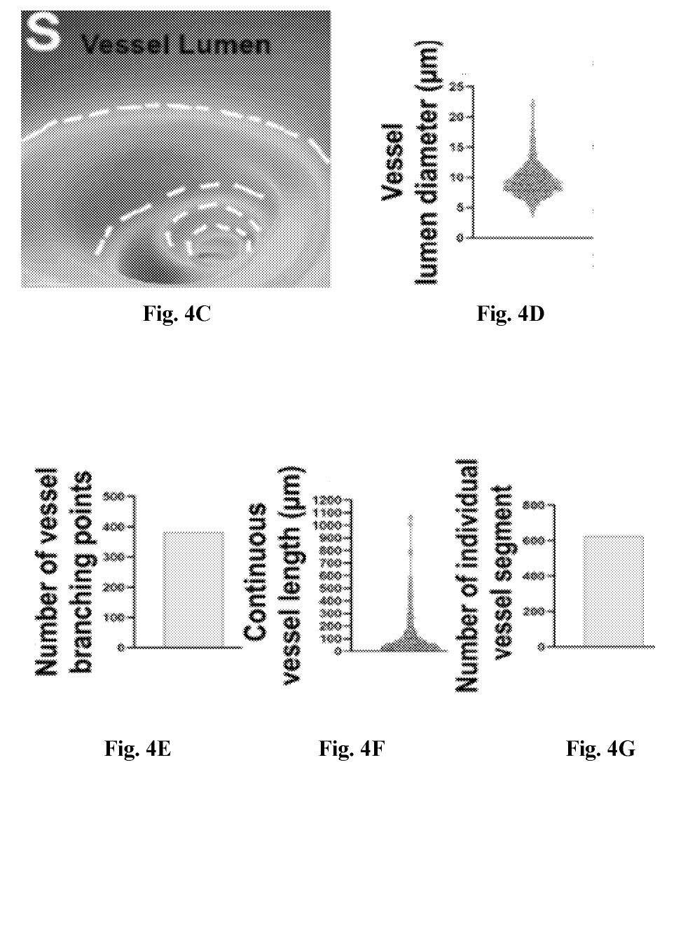

To visualize the three-dimensional vascular architecture, tissue clearing and lightsheet microscopy revealed an extensive, lumenized vascular network throughout the organoids (FIG. 4A). IMARIS-based 3D reconstruction confirmed well-organized, branching architecture (FIG. 4B), while cross-sectional imaging validated the presence of patent lumens (FIG. 4C). Quantitative morphometric analyses demonstrated progressive vascular maturation, as reflected by measurements of lumen diameter, branching complexity, vessel length, and segment number (FIGS. 4D-4G).

### Example 10: Tau-seeding in vascularized, immunocompetent cortical organoid leads to intracellular tau pathologies

To further confirm that the cortical organoids prepared herein behave like CNS tissues obtained from in vivo, the present study performed Tau-seeding assay in the cortical organoids. Tau-seeding assays are common performed using brain tissues obtained directly from animal subjects.

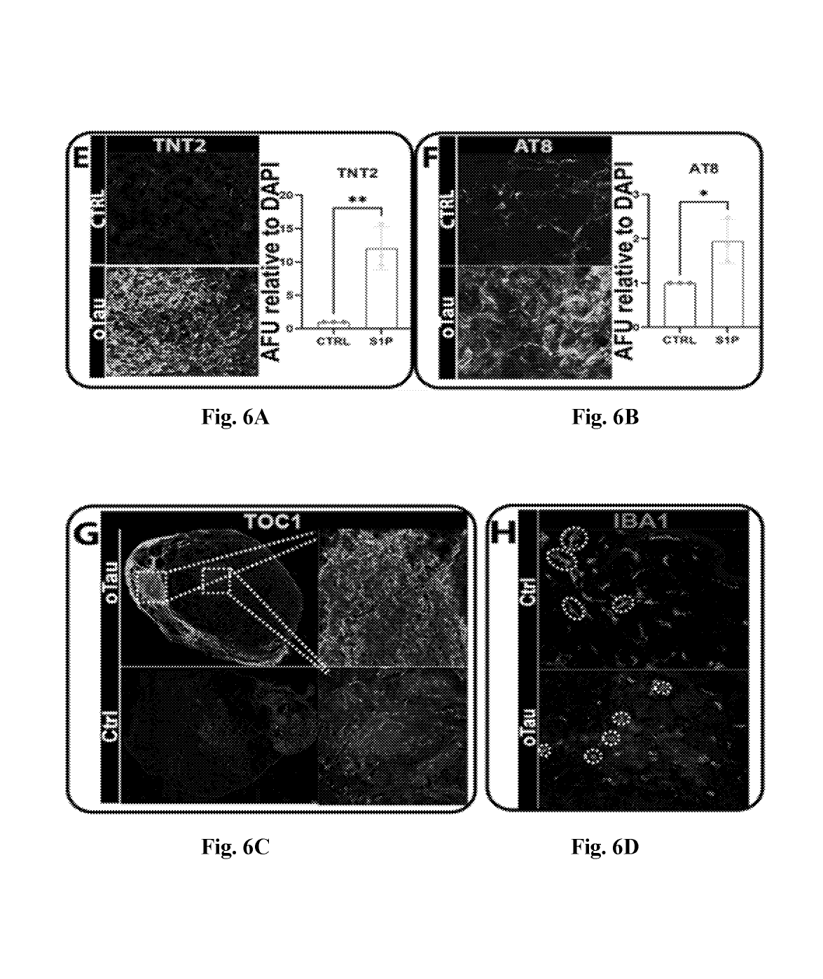

Robust tau pathologies were induced in vascularized, immunocompetent cortical organoids when seeded with oligomeric tau fibrils (oTau) isolated from postmortem AD patient brains (S1P, a mixture of oTau from multiple AD patients characterized and provided by the Wolozin Lab. After 3 weeks of exposure, tau-seeded organoids exhibited robust tau hyperphosphorylation and pathological conformations, as confirmed by AT8, TNT2, and TOC1 immunostaining (FIGS. 6A-6C). Accompanying these tau pathologies, activated tufted microglia were observed in tau-seeded organoids, contrasting sharply with the surveilling ramified microglia present in vehicle controls (FIG. 6D). This microglial activation indicates an inflammatory response to pathological tau accumulation within the organoid system, demonstrating immunocompetency of the organoids, as well as a high degree of similarity between the cortical organoids herein and real CNS tissues.

### Example 11

The present study establishes vascularized, immunocompetent cortical organoids (viCOs) as a next-generation human platform to interrogate neurovascular–neuroimmune interactions in development and disease. By integrating endothelial, mural, and microglial lineages derived from a single hiPSC source into a three-dimensional cortical scaffold, viCOs address long-standing limitations of conventional brain organoids, which lack intrinsic vasculature and resident microglia and therefore incompletely model blood–brain barrier (BBB) function, metabolic support, and neuroinflammation. The optimization of mesoderm–cortical integration parameters, biomaterial scaffolding, and cortical seeding density yielded constructs with balanced neuroectodermal, glial, vascular, and microglial compartments that approximate the composition of the developing human cortex, including CD31⁺ lumenized microvessels ensheathed by αSMA⁺/PDGFRβ⁺ mural cells and TMEM119⁺ microglia closely apposed to GFAP⁺ astrocytes, indicative of neurovascular unit assembly.

Single-cell RNA sequencing further demonstrated that viCOs support diverse neuronal, glial, endothelial, mural, and microglial populations with transcriptional signatures consistent with functional maturation, extending prior reports of vascularized cortical organoids and microglia-containing cerebral organoids that individually captured either vascular or immune elements, but not both in a syngeneic configuration. The presence of transcriptionally defined microglia within an internally vascularized environment is particularly relevant, as microglia dynamically sense changes in perfusion, barrier integrity, and extracellular milieu, and their state transitions are increasingly implicated as key drivers and modulators of neurodegeneration. The data suggest that the coordinated inclusion of these lineages within a unified hiPSC-derived system enables emergent properties—such as neurovascular unit formation and microglial surveillance of parenchymal and vascular compartments—that are difficult to reproduce in reductionist co-culture systems or non-vascularized organoids.

Challenging viCOs with AD-patient-derived oligomeric tau fibrils provided a stringent functional test of the model’s disease relevance. Tau seeding elicited hallmark features of human tauopathy, including robust tau hyperphosphorylation and aggregation, microglial activation with tufted morphologies, heightened inflammatory signaling, and evidence of neurodegeneration, aligning with observations that reactive microglia can amplify tau pathology and that neurovascular dysfunction and inflammation act as intertwined drivers of disease progression in vivo. Importantly, these responses emerged within a fully human, syngeneic context in which neurons, astrocytes, endothelial cells, mural cells, and microglia share an identical genetic background, minimizing confounds introduced by mixing primary or immortalized cells of disparate origins and enabling precise dissection of genotype-dependent effects. This feature distinguishes viCOs from prior organoid models incorporating exogenous endothelial or myeloid cells, which, while informative, are less suited to mechanistic genetics or patient-specific stratification.

The findings therefore position viCOs as a versatile microphysiological platform for probing the reciprocal interactions among tau pathology, BBB disruption, and neuroimmune activation. Because viCOs manifest lumenized vessels and mural coverage, they offer opportunities to investigate barrier permeability, transport, and perfusion-related stressors, and to model how vascular insults—hypoxia, hypertension-mimicking shear, or exposure to circulating inflammatory mediators—reshape microglial states and neuronal resilience. Likewise, the presence of resident microglia allows systematic interrogation of how microglial genotypes (for example, APOE, TREM2, or PLCG2 alleles) modulate tau propagation, and neurovascular coupling, building on emerging evidence that microglial genetic variants critically influence susceptibility to AD and related dementias. The modular nature of the approach should facilitate incorporation of patient-derived hiPSC lines bearing familial or sporadic AD risk variants, enabling individualized modeling of neurovascular–immune mechanisms and preclinical testing of targeted interventions.

In summary, viCOs close a critical gap between traditional brain organoids and the complex neurovascular–immune milieu of the human cortex by providing a syngeneic, vascularized, and microglia-containing 3D system that robustly models disease progression in neurological disorders.By enabling parallel readouts of neuronal integrity, vascular structure, and microglial state within a unified framework, this platform offers new opportunities to disentangle causal relationships among neurodegeneration, BBB breakdown, and inflammation, to identify cell type–specific vulnerabilities, and to evaluate candidate therapeutics that target the neurovascular unit or microglial signaling in neurological disorders. Looking forward, applying viCOs to diverse genetic backgrounds and environmental exposures should help illuminate convergent and divergent mechanisms in neurological disorders and inform the development of precision therapies that modulate neurovascular–immune networks rather than neurons alone.

### Enumerated Embodiments

In some aspects, the present invention is directed to the following non-limiting embodiments:

Embodiment 1: A method of preparing an organoid, the method comprising:
preparing a central nervous system (CNS) organoid by culturing first stem cells under a neuronal differentiation condition for a first duration of time,
optionally wherein the CNS organoid comprises neural progenitor cells, neurons,
or astrocytes;
preparing a mesoderm organoid by culturing second stem cells under a mesodermal differentiation condition for a second duration of time;
dissociating the cells in the CNS organoid to obtain a plurality of dissociated CNS cells; and
co-culturing the plurality of dissociated CNS cells with the mesoderm organoid under a co-culture condition.

Embodiment 2: The method of Embodiment 1, wherein the first stem cells and the second stem cells are the same type of stem cells, optionally wherein the first stem cells and the second stem cells are from the same stem cell source.

Embodiment 3: The method of Embodiment 1, wherein the first stem cells or the second stem cells are independently induced pluripotent stem cells (iPSCs), optionally wherein the first stem cells or the second stem cells are independently human iPSCs (hiPSCs).

Embodiment 4: The method of Embodiment 1, wherein at least one of the following applies:
(a) the first duration of time is longer than the second duration of time,
(b) the first duration of time ranges from about 18 days to about 24 days,
(c) the second duration of time ranges from about 10 days to about 15 days.

Embodiment 5: The method of Embodiment 1, wherein co-culturing the plurality of dissociated CNS cells with the mesoderm organoid comprising co-culturing a predetermined number of the dissociated CNS cells and a predetermined number of the mesoderm organoid, such that the ratio between the dissociated CNS cells and the mesoderm organoid allows ratios between neural progenitors, astrocytes, blood vessel endothelial cells, and microglia cells are similar to the ratios in a brain tissue from an in vivo source.

Embodiment 6: The method of Embodiment 1, wherein co-culturing the plurality of dissociated CNS cells with the mesoderm organoid comprising co-culturing about 80,000 to about 150,000 dissociated CNS cells with one mesoderm organoid.

Embodiment 7: The method of Embodiment 1, wherein the plurality of dissociated CNS cells and the mesoderm organoid are co-cultured in a biomaterial scaffold.

Embodiment 8: The method of Embodiment 1, wherein at least one of the following applies:
(a) the neuronal differentiation condition comprises treatment with dorsomorphin (DM), SB431542, EGF, or FGF;
(b) the mesodermal differentiation condition comprises treatment with CHIR99021, bFGF, BMP4, or VEGF;
(c) the co-culture condition comprises treatment with BDNF, NT3, VEGF, IL-34, or M-CSF.

Embodiment 9: An organoid prepared according to the method of Embodiment 1.

Embodiment 10: An organoid, comprising:
neurons or neural progenitor cells (NPCs);
microglia cells,
   optionally wherein the microglia cells comprise TMEM119-expressing cells; and a microvascular network,
   optionally wherein the microvascular network comprises CD31-expressing
   endothelial cells.

Embodiment 11: The organoid of Embodiment 10, wherein at least one of the following applies:
(a) the microglia cells are immunocompetent,
(b) the microglia cells are in a resting, ramified state when the organoid is in a homeostatic state,
(c) the microglia are activated when the organoid is in an inflamed state,
   optionally the microglia are capable of activation when the organoid challenged
   by oligomeric Tau.

optionally the microglia are capable of activation when the organoid challenged by oligomeric Tau.

Embodiment 12: The organoid of Embodiment 10, wherein the microvascular network comprises both endothelial cells and mural cells.

Embodiment 13: The organoid of Embodiment 10, wherein at least one of the following applies:
(a) the organoid comprises a blood brain barrier (BBB),
(b) the organoid comprises pericytes or cells expressing PDGFRβ along the microvascular network,
(c) the organoid comprises astrocytes or cells expressing GFAP along the microvascular network.

Embodiment 14: The organoid of Embodiment 10, which is perfusable.

Embodiment 15: The organoid of Embodiment 10, wherein all or some of the cells in the organoid are differentiated from induced pluripotent stem cells (iPSCs), optionally wherein all the cells in the organoid are differentiated from a single source of iPSCs.

Embodiment 16: The organoid of Embodiment 15, wherein the iPSCs are human iPSCs (hiPSCs).

Embodiment 17: The organoid of Embodiment 10, wherein at least one of the following applies:
(a) the amount of the SOX2⁺ cells ranges from about 5% to about 70% based on a total number of cells,
(b) the area occupied by the GFAP⁺ cells ranges from about 10% to about 80% based on a total area of the organoid,
(c) the area occupied by the TMEM119⁺ cells ranges from about 2% to about 10% based on a total area of the organoid,
(d) the CD31⁺ vessel‑like area ranges from about 5% to about 20% based on a total area of the organoid

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method of preparing an organoid, the method comprising:
preparing a central nervous system (CNS) organoid by culturing first stem cells under a neuronal differentiation condition for a first duration of time,
optionally wherein the CNS organoid comprises neural progenitor cells, neurons, or astrocytes;
preparing a mesoderm organoid by culturing second stem cells under a mesodermal differentiation condition for a second duration of time;
dissociating the cells in the CNS organoid to obtain a plurality of dissociated CNS cells; and
co-culturing the plurality of dissociated CNS cells with the mesoderm organoid under a co-culture condition.

2. The method of claim 1, wherein the first stem cells and the second stem cells are the same type of stem cells, optionally wherein the first stem cells and the second stem cells are from the same stem cell source.

3. The method of claim 1, wherein the first stem cells or the second stem cells are independently induced pluripotent stem cells (iPSCs), optionally wherein the first stem cells or the second stem cells are independently human iPSCs (hiPSCs).

4. The method of claim 1, wherein at least one of the following applies:
(a) the first duration of time is longer than the second duration of time,
(b) the first duration of time ranges from about 18 days to about 24 days,
(c) the second duration of time ranges from about 10 days to about 15 days.

5. The method of claim 1, wherein co-culturing the plurality of dissociated CNS cells with the mesoderm organoid comprising co-culturing a predetermined number of the dissociated CNS cells and a predetermined number of the mesoderm organoid, such that the ratio between the dissociated CNS cells and the mesoderm organoid allows ratios between neural progenitors, astrocytes, blood vessel endothelial cells, and microglia cells are similar to the ratios in a brain tissue from an in vivo source.

6. The method of claim 1, wherein co-culturing the plurality of dissociated CNS cells with the mesoderm organoid comprising co-culturing about 80,000 to about 150,000 dissociated CNS cells with one mesoderm organoid.

7. The method of claim 1, wherein the plurality of dissociated CNS cells and the mesoderm organoid are co-cultured in a biomaterial scaffold.

8. The method of claim 1, wherein at least one of the following applies:
(a) the neuronal differentiation condition comprises treatment with dorsomorphin (DM), SB431542, EGF, or FGF;
(b) the mesodermal differentiation condition comprises treatment with CHIR99021, bFGF, BMP4, or VEGF;
(c) the co-culture condition comprises treatment with BDNF, NT3, VEGF, IL-34, or M-CSF.

9. An organoid prepared according to the method of claim 1.

10. An organoid, comprising:
neurons or neural progenitor cells (NPCs);
microglia cells,
optionally wherein the microglia cells comprise TMEM119-expressing cells; and
a microvascular network,
optionally wherein the microvascular network comprises CD31-expressing endothelial cells.

11. The organoid of claim 10, wherein at least one of the following applies:
(a) the microglia cells are immunocompetent,
(b) the microglia cells are in a resting, ramified state when the organoid is in a homeostatic state,
(c) the microglia are activated when the organoid is in an inflamed state,
optionally the microglia are capable of activation when the organoid challenged by oligomeric Tau.

12. The organoid of claim 10, wherein the microvascular network comprises both endothelial cells and mural cells.

13. The organoid of claim 10, wherein at least one of the following applies:
(a) the organoid comprises a blood brain barrier (BBB),
(b) the organoid comprises pericytes or cells expressing PDGFRβ along the microvascular network,
(c) the organoid comprises astrocytes or cells expressing GFAP along the microvascular network.

14. The organoid of claim 10, which is perfusable.

15. The organoid of claim 10, wherein all or some of the cells in the organoid are differentiated from induced pluripotent stem cells (iPSCs), optionally wherein all the cells in the organoid are differentiated from a single source of iPSCs.

16. The organoid of claim 15, wherein the iPSCs are human iPSCs (hiPSCs).

17. The organoid of claim 10, wherein at least one of the following applies:
(a) the amount of the SOX2⁺ cells ranges from about 5% to about 70% based on a total number of cells,
(b) the area occupied by the GFAP⁺ cells ranges from about 10% to about 80% based on a total area of the organoid,
(c) the area occupied by the TMEM119⁺ cells ranges from about 2% to about 10% based on a total area of the organoid,
(d) the CD31⁺ vessel‑like area ranges from about 5% to about 20% based on a total area of the organoid.
